(19) United States
(12) Patent Application Publication
(10) Pub. No.: US 2004/0023986 A1
(43) Pub. Date: Feb. 5, 2004

(54) Substituted felbamate derived compounds

(76) Inventor: Timothy L. Macdonald, Charlottesville, VA (US)

Correspondence Address: UNIVERSITY OF VIRGINIA PATENT FOUNDATION, 1224 WEST MAIN STREET, SUITE 1-110, CHARLOTTESVILLE, VA, 22903, US

(21) Appl. No.: 10/415,167

(22) PCT Filed: Oct. 23, 2001
(86) PCT No.: PCT/US01/47665

(51) Int. Cl. A61K 31/505; A61K 31/44; A61K 31/415

(52) U.S. Cl. 514/256; 514/406; 514/341

(57) ABSTRACT

The present invention relates to novel felbamate derivatives and their use to threat neurological diseases such as epilepsy and neuropathic pain, and to treat tissue damage resulting form ischemic events. The felbamate derivatives are modified to prevent the formation of metabolites that are believed responsible for the toxicity associated with felbamate therapy.

This application claims priority under 35 U.S.C. §119(e) to provisional patent application No. 60/243,023 filed Oct. 25, 2000 and 60/243,024 filed Oct. 25, 2000, the disclosures of which are incorporated herein.

### FIELD OF THE INVENTION

The present invention is directed to novel derivatives of 2-phenyl-1,3-propanediol dicarbamate (felbamate), and the use of such derivatives as therapeutic agents. More particularly, compositions comprising the present felbamate derivatives can be administered for treating a variety of neurological related maladies such as reducing the incidence and severity of epileptic seizures and for preventing and treating hypoxic damage resulting from an ischemnic event.

### BACKGROUND OF THE INVENTION

Felbamate (2-phenyl-1,3-propanediol dicarbamate) is a known pharmaceutical compound having been described in U.S. Pat. Nos. 2,884,444 and 4,868,327, the disclosures of which are expressly incorporated herein. Felbamate is a modulator of NMDA (N-methyl-D-aspartate) receptor function, and a glycine site antagonist but also has other reported mechanisms of actions.

Felbamate has also been reported to interact at the AMPA/kainate receptor, facilitate the function of the GABA receptor, and modulate Na.sup.+ channel conductance. Felbamate has also been demonstrated to decrease delayed neuronal cell death after kainic acid induced status epilepticus in animals. Glycine or d-serine were able to functionally reverse the anticonvulsant and ischemic protective effect of felbamate.

Felbamate has been proposed for use in treating various neurological disorders including the control of epileptic seizures. For example, U.S. Pat. No. 4,978,680 discloses the use of felbamate for the prevention and control of epileptic seizures; U.S. Pat. No. 5,082,861 relates to the use of felbamate for the prevention and control of epileptic seizures associated with complex partial seizures; and U.S. Pat. No. 5,292,772 relates to the use of felbamate for the prevention and control of epileptic seizures associated with Lennox-Gastaut syndrome. The disclosures of U.S. Pat. Nos. 4,978,680, 5,082,861 and 5,292,772 are expressly incorporated herein.

Felbamate has also been reported to have efficacy in reducing cellular damage resulting from vascular reperfusion (U.S. Pat. No. 5,462,966) and preventing and treating tissue damage resulting from an ischemic event (U.S. Pat. No. 5,055,489). For example, compositions comprising felbamate can be administered to control or prevent hypoxic damage resulting from stroke and other cerebral ischemic events. The disclosure of U.S. Pat. Nos. 5,462,966 and 5,055,489 are also expressly incorporated herein.

Felbamate was approved in July 1993 for the treatment of several forms of epilepsy. Felbamate demonstrated an excellent therapeutic index throughout preclinical and clinical trials with only relatively mild side effects observed and/or reported. In its first year of approval, between 100,000 and 125,000 patients were placed on felbamate therapy in the U.S. However, within the first year of felbamate's wide spread use, adverse reactions were reported, notably aplastic anemia and hepatotoxicity. (See Pennell et al., Neurology. 45, 456-460 (1995) and O'Neil et al., Neurology. 46, 1457-1459 (1996)). The severity and frequency of occurrence of these side effects prompted a recommendation by the FDA in August 1994 to withdraw patients from felbamate therapy, unless the benefit of seizure control outweighed the risk of the reported toxicities.

The present invention is directed to felbamate derivatives, and their metabolites, that exhibit therapeutic properties similar to felbamate, without the adverse reactions observed with felbamate administration. In accordance with the present invention these felbamate derivatives are used to treat neurological disorders including and prevent and/or control tissue damage resulting from hypoxic conditions. More particularly, the present novel compounds are believed to be useful for treating epileptic seizures, acute and chronic neurogenerative conditions, neuropsychiatric disorders, pain and for preventing or alleviating cellular damaged caused by myocardial or cerebral ischemic events.

The presently disclosed derivatives of felbamate are believed to have biological activities similar to those of the parent felbamate compound. However, the present compounds have been modified to prevent the formation of metabolites that are believed to cause the adverse reactions associated with the use of felbamate. Accordingly, it is anticipated that the felbamate derivatives of the present invention can be substituted for felbamate for all the therapeutic uses that have been proposed for felbamate. In addition, many of the derivatives have enhanced activities allowing for the administration of lower therapeutically effective dosage forms.

### SUMMARY OF THE INVENTION

The present invention is directed to novel compounds of the general formula:

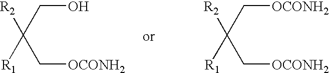

wherein R₂ is halo and R₁ is selected from the group consisting of C₁-C₉ alkyl, C₃-C₉ cycloalkyl, alkylated C₃-C₉ cycloalkyl, 2-thienyl, 3-thienyl, 2-pyridinyl, 3-pyridinyl, 4-pyridinyl, 2-(1,3 diazinyl), 4-(1,3 diazinyl), 5-(1,3 diazinyl), 2-(1, 4 diazinyl), 2-imidazoyl, 4-imidazoyl, 2-(1, 3 oxazinyl), 4-(1, 3 oxazinyl), 5-(1, 3 oxazinyl), 2-(thiazinyl), 4-(thiazinyl), 5-(thiazinyl) and

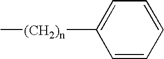

wherein n is 1-3, and the use of such compounds to provide relief from a number of neurological conditions.

### DETAILED DESCRIPTION OF THE INVENTION

Definitions

In describing and claiming the invention, the following terminology will be used in accordance with the definitions set forth below.

As used herein, the term “purified” and like terms relate to the isolation of a molecule or compound in a form that is substantially free of contaminants normally associated with the molecule or compound in a native or natural environment.

As used herein, the term “pharmaceutically acceptable carrier” encompasses any of the standard pharmaceutical carriers, such as a phosphate buffered saline solution, water and emulsions such as an oil/water or water/oil emulsion, and various types of wetting agents.

As used herein, “effective amount” means an amount sufficient to produce a selected effect. For example, an effective amount of a neuroprotective felbamate derivative is an amount of the active agent sufficient to significantly reduce the incidence and severity of epileptic seizures. An effective amount of a hypoxia ameliorating felbamate derivative is an amount of the active agent sufficient to prevent or significantly reduce cellular damage resulting from coronary artery occlusion/reperfusion or other hypoxia inducing event.

The term, “parenteral” means not through the alimentary canal but by some other route such as subcutaneous, intramuscular, intraspinal, or intravenous.

As used herein, the term “treating” includes prophylaxis of the specific disorder or condition, or alleviation of the symptoms associated with a specific disorder or condition and/or preventing or eliminating said symptoms.

The general chemical terms used in the description of the compounds of the present invention have their usual meanings. For example, the term “alkyl” by itself or as part of another substituent means a straight or branched aliphatic chain having the stated number of carbon atoms.

As used herein, the term “halogen” or “halo” means Cl, Br, F, and I. The term “haloalkyl” as used herein refers to a C₁-C₄ alkyl radical bearing at least one halogen substituent, for example, chloromethyl, fluoroethyl or trifluoromethyl and the like.

The term “C₁-Cₙ alkyl” wherein n is an integer, as used herein, represents a branched or linear alkyl group having from one to the specified number of carbon atoms. Typically C₁-C₆ alkyl groups include, but are not limited to, methyl, ethyl, n-propyl, iso-propyl, butyl, iso-butyl, sec-butyl, tert-butyl, pentyl, hexyl and the like.

The term “C₂-Cₙ alkenyl” wherein n is an integer, as used herein, represents an olefinically unsaturated branched or linear group having from 2 to the specified number of carbon atoms and at least one double bond. Examples of such groups include, but are not limited to, 1-propenyl, 2-propenyl, 1,3-butadienyl, 1-butenyl, hexenyl, pentenyl, and the like.

The term “C₂-Cₙ alkynyl” wherein n is an integer refers to an unsaturated branched or linear group having from 2 to the specified number of carbon atoms and at least one triple bond. Examples of such groups include, but are not limited to, 1-propynyl, 2-propynyl, 1-butynyl, 2-butynyl, 1-pentynyl, and the like.

The term “C₃-Cₙ cycloalkyl” wherein n=4-8, represents cyclopropyl, cyclobutyl, cyclopentyl, cyclohexyl, cycloheptyl, and cyclooctyl.

As used herein, the term “optionally substituted” refers to from zero to four substituents, wherein the substituents are each independently selected. More preferredly, the term refers to from zero to three independently selected substituents. Each of the independently selected substituents may be the same or different than other substituents.

As used herein the term “aryl” refers to a mono- or bicyclic carbocyclic ring system having one or two aromatic rings including, but not limited to, phenyl, benzyl, naphthyl, tetrahydronaphthyl, indanyl, indenyl, and the like. Substituted aryl includes aryl compounds having one or two C₁-C₆ alkyl, halo or amino substituents. The term (C₅-C₈ alkyl)aryl refers to any aryl group which is attached to the parent moiety via the alkyl group.

The term “heterocyclic group” refers to a C₃-C₈ cycloalkyl group containing from one to three heteroatoms wherein the heteroatoms are selected from the group consisting of oxygen, sulfur, and nitrogen.

The term “bicyclic” represents either an unsaturated or saturated stable 7-to 12-membered bridged or fused bicyclic carbon ring. The bicyclic ring may be attached at any carbon atom which affords a stable structure. The term includes, but is not limited to, naphthyl, dicyclohexyl, dicyclohexenyl, and the like.

Any ring structure drawn with one or more bonds extending from the center of the ring is intended to designate a series of compounds that have a bond(s) extending from one of the carbon atoms of the ring to another atom. For example, the structure:

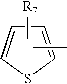

designates a series of compounds including 2-thienyl or 3-thienyl groups that contain an R₇ substituent at one of the remaining ring carbon atoms.

As used herein the term “neurological disease” or “neurological condition” includes neurological related maladies such as spasticity, depression or mood disorders, neuropathic pain, Alzheimer's Disease, Parkinson's Disease, HIV Dementia and neurological disorders that involve excessive activation of the N-methyl-D-aspartate (NMDA) receptor.

The Invention

The novel compounds of the present invention are derivatives of felbamate. In particular, the original felbamate structure has been modified to replace the phenyl moiety with a substituted or unsubstituted heterocyclic group, or a substituted or unsubstituted alkyl side chain. These derivatives are further modified in accordance with the invention to prevent the formation of metabolite 2-Z-propenyl upon administration to a warm blooded vertebrate (wherein Z is a heterocyclic group or alkyl side chain). The formation of 2-phenylpropenyl (atropaldehyde) is believed to be responsible for the adverse effects associated with the administration of felbamate, and thus the formation of similar 2-Z-propenyl metabolites from the present felbamate derivatives are anticipated to cause similar adverse effects.

The following metabolic pathway (Scheme I) of felbamate (1) has been proposed to produce 3-carbamoyl-2-phenylpropionaldehyde (3). 3-Carbamoyl-2-phenylpropionaldehyde (3) is believed to be a reactive intermediate in the oxidation of 2-phenyl-1,3-propanediol monocarbamate (2) to the major human metabolite 3-carbamoyl-2-phenylpropionic acid (4). In addition, the aldehyde carbamate (3) was found to undergo spontaneous elimination to form the α,β unsaturated aldehyde, 2-phenylpropenal (5), commonly known as atropaldehyde. Atropaldehyde has been proposed to play a role in the development of toxicity during felbamate therapy.

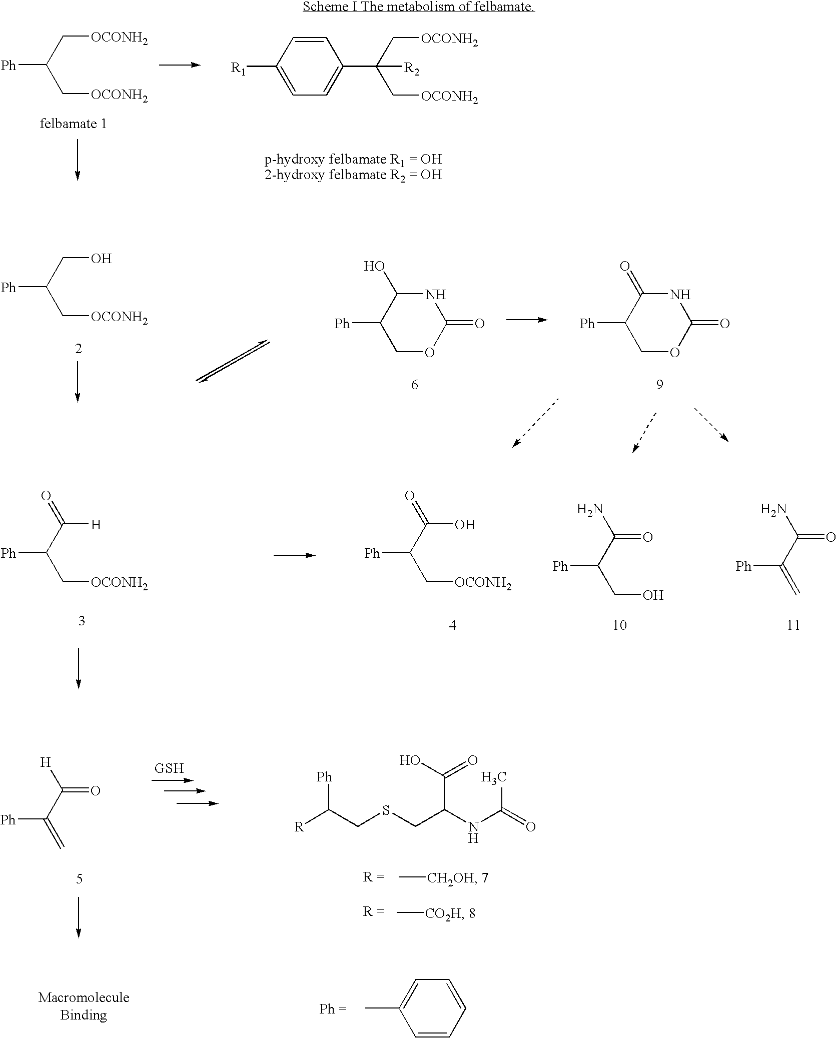

Evidence for atropaldehyde formation in vivo has been reported with the identification of modified N-acetyl-cysteine conjugates 7 and 8 of atropaldehyde in both human and rat urine after felbamate administration. Identification of the atropaldehyde derived mercapturic acids in urine after felbamate administration is consistent with the hypothesis that atropaldehyde is formed in vivo and that it reacts with thiol nucleophiles.

Based on the hypothesis that the toxicity associated with felbamate administration is directly correlated to the amount of atropaldehyde formed, the present invention is directed to the development of a new class of agents structurally related to felbamate that cannot undergo metabolism to form compounds structurally similar to atropaldehyde. In accordance with the present invention the phenyl group of felbamate is replaced with a heterocyclic group or an alkyl chain (straight or branched) and the benzylic hydrogen of felbamate (1) is replaced with a substituent “X” as shown in the following metabolic scheme (Scheme II). X is halo, more preferably Cl or F, and in one preferred embodiment the substituent is a fluorine atom.

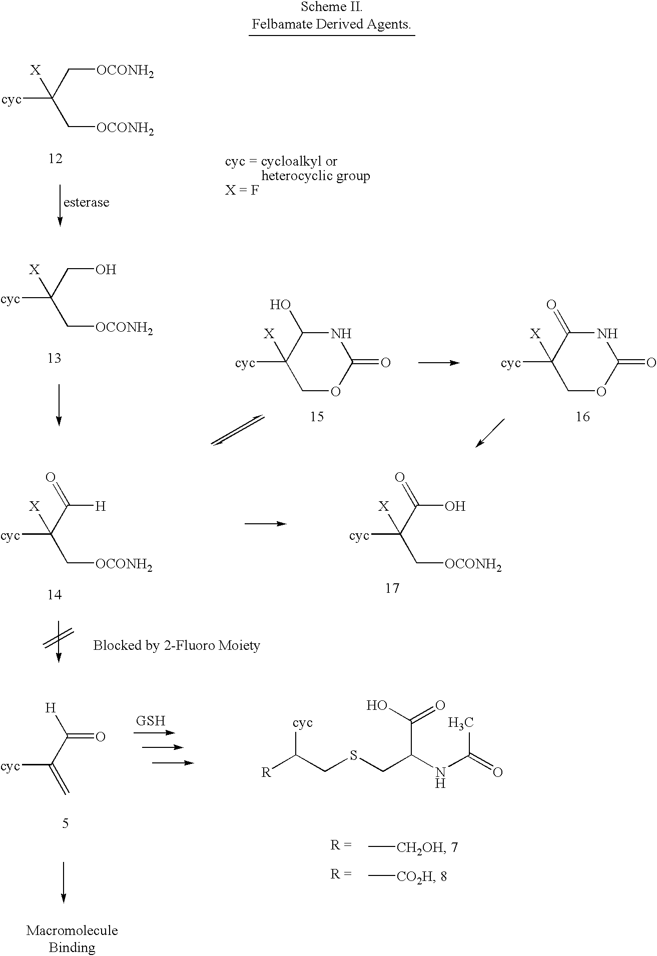

2-Fluoro-2-Z-1, 3-propanediol dicarbamate (12) and 2-Fluoro-2-Z-1, 3-propanediol monocarbamate fluoro monocarbamate felbamate (13) are derivatives of known antiepileptic agents. These agents represent a new class of anti-epileptics that, while possessing structural similarity to felbamate, will not exhibit felbamate's metabolic profile and will not induce adverse reactions such as those associated with the use of felbamate.

The present invention also encompasses the derivatives of the potentially active metabolites of the present felbamate derivatives. In particular this includes fluoro oxazinane-dione 16 and difluoro oxazinane-dione, which are derivatives of the felbamate metabolite oxazinane-dione 9. The structure of oxazinane-dione 9 bears intriguing similarity to several established anti-epileptic drugs including phenobarbital, phenytoin, oxazinane-dione, metharbital and ethotoin, as illustrated below:

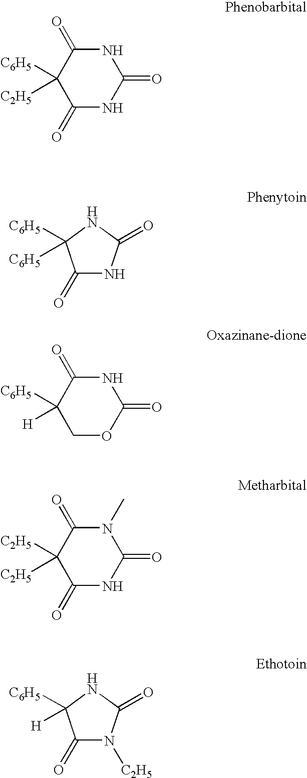

It is anticipated, therefore, that the oxazinane-dione 9 is responsible for some aspects of felbamate's efficacy in vivo. As patients undergoing felbamate therapy for seizure control ingest large quantities of felbamate (grams per day), even a 1-2% conversion to a pharmacologically active metabolite could have significant effects. This could play an important role in the seizure control observed with felbamate, particularly if the metabolite (i.e., the oxazinane-dione) was a more potent compound. In light of the possibility that the oxazinane-dione 9 could be a metabolic precursor to the major human metabolite, acid carbamate 4, it may be formed in significant quantities (the acid carbamate was reported to represent˜12% of a dose). Because the parent oxazinane-dione 9 has been found to be unstable at relevant pH, oxazinane-diones 16 are considered candidates for development as potential antiepileptic agents.

In accordance with one embodiment, the present invention is directed to a compound having the general structure:

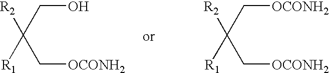

wherein R₁ is selected from the group consisting of selected from the group consisting of C₁-C₉ alkyl, C₃-C₉ cycloalkyl, C₁-C₉ alkylated C₃-C₉ cycloalkyl,

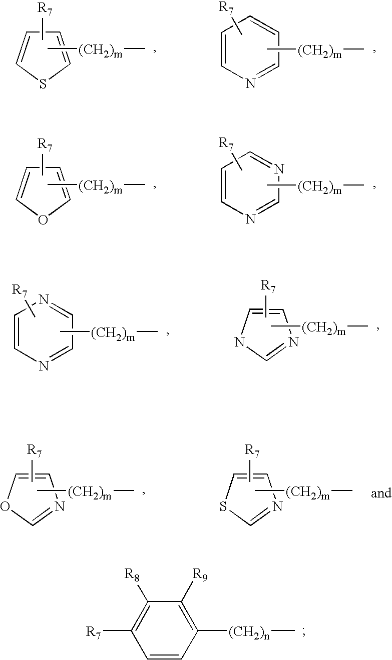

R₂ is F or Cl, m is 0-3, n is 1-3 and R₇, R₈ and R₉ are independently selected from the group consisting of H, halo, alkyl, haloalkyl and hydroxy.

In one embodiment the compounds has the general formula

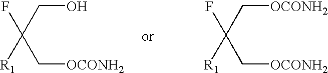

wherein R₁ is selected from the group consisting of selected from the group consisting of C₁-C₉ alkyl, C₃-C₉ cycloaLkyl, C₁-C₉ alkylated C₃-C₉ cycloalkyl,

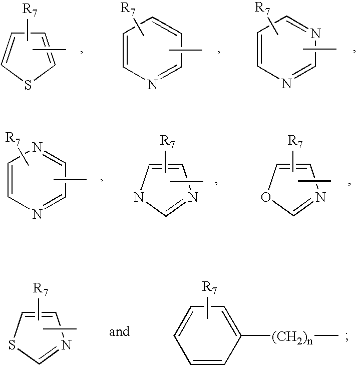

n is 1-3 and R₇ is selected from the group consisting of H, halo, alkyl, haloalkyl and hydroxy. In one preferred embodiment R₇ is H.

One preferred set of compounds includes felbamate derivatives having the general formula:

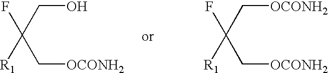

wherein R₁ is selected from the group consisting of C₁-C₉ alkyl, C₃-C₉ cycloalkyl, C₁-C₉ alkylated C₃-C₉ cycloalkyl, 2-thienyl, 3-thienyl, 2-pyridinyl, 3-pyridinyl, 4-pyridinyl, 2-(1,3 diazinyl), 4-(1,3 diazinyl), 5-(1,3 diazinyl), 2-(1, 4 diazinyl), 2-imidazoyl, 4-imidazoyl, 2-(1, 3 oxazinyl), 4-(1, 3 oxazinyl), 5-(1, 3 oxazinyl), 2-(thiazinyl), 4-(thiazinyl), 5-(thiazinyl) and

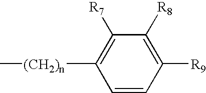

wherein n is 1-3 and R₇, R₈ and R₉ are independently selected from the group consisting of H, halo, C₁-C₄ alkyl, C₁-C₄ haloalkyl and hydroxy.

Compositions comprising the felbamate derivative of the present invention can be used to treat patients suffering from a neurological diseases such as epileptic seizures or can be used to prevent or treat reperfusion injuries resulting from stroke, myocardial infarction, and spinal chord perfusion-type injuries. It is believed that the felbamate derivatives of the present invention also have utility for treating conditions characterized by the presence of reactive oxygen species (ROS).

The felbamate derivative of the present invention can be combined with pharmaceutically acceptable carriers, stabilizing agents, solubilizing agents, and fillers known to those skilled in the art for administration to the patient. The compositions can be formulated using standard delivery vehicles and standard formulations for oral, parenteral or transdermal delivery.

In an alternative embodiment the present invention is also directed to cyclic derivatives of felbamate, wherein the compound has the general structure:

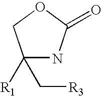

wherein R₁ is selected from the group consisting of selected from the group consisting of C₁-C₉ alkyl, C₃-C₉ cycloalkyl, C₁-C₉ alkylated C₃-C₉ cycloalkyl,

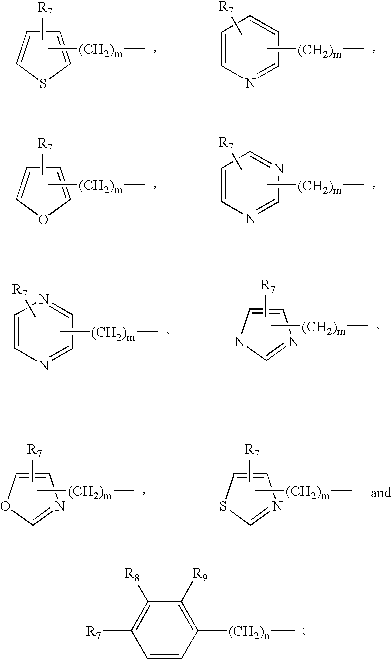

R₃ is hydroxy or —OCONH₂, m is 0-3, n is 1-3 and R₇, R₈ and R₉ are independently selected from the group consisting of H, halo, alkyl, haloalkyl and hydroxy. In one embodiment, m is 0, R₇ is H and R₃ is —OCONH₂.

In accordance with one embodiment the felbamate derivative has the general structure:

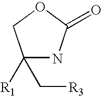

wherein R₃ is hydroxy or —OCONH₂ and R₁ is selected from the group consisting of C₁-C₉ alkyl, C₃-C₉ cycloalkyl, C₁-C₉ alkylated C₃-C₉ cycloalkyl, C₃-C₉ cycloalkyl, 2-thienyl, 3-thienyl, 2-pyridinyl, 3-pyridinyl, 4-pyridinyl, 2-(1,3 diazinyl), 4-(1,3 diazinyl), 5-(1,3 diazinyl), 2-(1, 4 diazinyl), 2-imidazoyl, 4-imidazoyl, 2-(1, 3 oxazinyl), 4-(1, 3 oxazinyl), 5-(1, 3 oxazinyl), 2-(thiazinyl), 4-(thiazinyl), 5-(thiazinyl) and

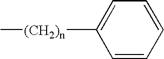

wherein n is 1-3.

In accordance with one embodiment a patient suffering from a neurological disorder (and in particular, neurological disorders that involve excessive activation of the NMDA receptor) or from a reperfusion injury can be treated with the felbamate derivatives, and/or the metabolites of the felbamate derivatives to alleviate the symptoms associated with said disorder or injury.

It is anticipated that the present felbamate derivatives will have similar activity to felbamate and therefore the present derivatives are anticipated to be efficacious in treating the disorders where felbamate is therapeutically effective. More particularly, U.S. Pat. Nos. 5,942,540 and 5,728,728, the disclosures of which are expressly incorporated herein, describe a number of diseases or conditions that can be treated by the administration of felbamate. The felbamate derivatives of the present invention are also useful in treating these diseases.

Obesity is a common human disorder which affects 10-15% of the population, of which up to 5% may be severely obese. It is estimated that the mortality from obesity is between 300,000 to 400,000 per annum. Obesity is commonly measured by the BMI (body mass index) which is the weight in kilograms divided by the height in meters squared. The degree of obesity is determined by comparisons against standard deviations above the means for males and females.

The etiology of obesity is unknown but occurs when energy intake exceeds energy expenditure. Appetite is controlled by the ventromedial hypothalamus and complex interconnections with the limbic system and other portions of the brain. Recent neurochemical research has implicated leptin, GLP-1 (glucagon-like peptide 1) and neuropeptide-Y in the control of appetite. Leptin is a natural appetite suppressant which is released from adipose cells, travels to the brain and appears to exert some control over appetite and long term weight control. A defective leptin receptor has been postulated to be involved in obese patients. GLP-1, a brain hormone which promotes satiety, is believed to regulate short-term appetite. Neuropeptide-Y is a potent stimulator of appetite whose effects can be blocked by leptin and GLP-1. The complete interaction of these compounds remains to be elucidated.

Felbamate has been known to induce weight loss in patients treated with epilepsy. In a study of felbamate as add-on therapy in partial epilepsy in children, weight loss was transient and returned to normal after twenty weeks (Carmant J., Pediatr 125:481-486, 1994). Weight loss of 4-5% was noted in patients on felbamate monotherapy (Faught E., Neurology 43:688-692, 1993). It has been suggest that weight loss from felbamate is due to NMDA receptor modulation in the hypothalamic structures involved in appetite control.

Felbamate, administered chronically to humans in oral doses of from about 100-15,000 mg/day, advantageously from about 1200-7200 mg/day (serum levels ranging from about 25-300 ug/ml), is efficacious in producing weight loss in obesity, type-II diabetes, and other genetic obesity disorders. It is anticipated that administration of the felbamate derivatives of the present invention art similar dosage ranges will similarly be efficacious in producing weight loss in individuals. Therefore, in accordance with one embodiment of the present invention the felbamate derivatives of the present invention are formulated as pharmaceutical compositions and administered to individuals to induce weight loss in those individuals.

Spasticity is a human motor disorder manifested by an increase in muscle tone and an exaggeration of deep tendon reflexes due to lesions of the corticospinal system. The spasticity is proportional to the rate and degree of stretch placed on the muscle. The most common causes are multiple sclerosis and spinal cord injury. Spasticity produces multiple medical complications, pain, and depression.

The etiology of spasticity is a decrease or malfunctioning of inhibitory mechanisms in the spinal cord leading to hyper-excitability of the tonic stretch and other reflexes. The mechanism may involve a decrease in both presynaptic GABA-ergic inhibition and postsynaptic inhibition. Noradrenergic receptors become supersensitive distal to spinal cord injury which is the rationale for using alpha-2 agonists as a treatment in spinal cord injury. GABA and glycine are the main inhibitory neurotransmitters in the spinal cord. Glycine acts at both the strychnine-insensitive and strychnine-sensitive receptors, the latter being more common in the spinal cord.

The pharmacotherapy of spasticity is directed at the potentiation of inhibitory transmission within the spinal cord, such as the mediation of presynaptic inhibition by GABA. Excitatory amino acids (EAA) increase spasticity and non-competitive NMDA antagonists depress spinal polysynaptic reflexes by inhibiting the release of EAA (Schwarz M., In Thilman AF, Ed. Spasticity, pp 85-97, 1993). Felbamate has both GABA enhancing properties and glycine-site strychnine insensitive antagonist properties which suggests efficacy in the treatment of spasticity.

Felbamate, administered chronically in oral doses of from about 100-15,000 mg/day, advantageously from about 1200-7200 mg/day (serum levels ranging from 25-300 ug/ml), is efficacious in reducing spasticity from both supraspinal and spinal lesions. Accordingly it is anticipated that administration of the felbamate derivatives of the present invention art similar dosage ranges will also alleviate spasticity from both supraspinal and spinal lesions.

Depression or mood disorders are psychopathologic states in which a disturbance of mood is either a primary determinant or constitutes the core manifestation. Secondary depression is an affective disorder caused by a systemic or neurological disease. Examples of neurologic diseases include but are not limited to multiple sclerosis, Parkinson's disease, head trauma, cerebral tumors, post-stroke, early dementing illness, and sleep apnea, while systemic diseases include infections, endocrine disorders, collagen vascular diseases, nutritional deficiencies and neoplastic disease. Secondary depression is common in post-myocardial infarct patients and carry a mortality three times that of non-depressed post-myocardial patients.

Felbamate, administered chronically in oral doses of from about 100 -15,000 mg/day, advantageously from about 1200-7200 mg/day (serum levels ranging from 25-300 ug/ml), is efficacious in reducing secondary symptomatic depression in both human systemic and neurologic diseases. Accordingly it is anticipated that administration of the felbamate derivatives of the present invention art similar dosage ranges will also alleviate secondary symptomatic depression in both human systemic and neurologic diseases.

In accordance with one embodiment the felbamate derivatives of the present invention are used to alleviate pain and in particular pain of neuropathic origin. Neuropathic pain is a chronic condition in which NMDA receptors in neural pain pathways have become “kindled” to an abnormally high level of sensitivity so that they spontaneously convey nerve messages that the patient perceives as pain even though no painful stimulus has been inflicted (see U.S. Pat. No. 5,925,634, the disclosure of which is incorporated herein). Excessive activation of NMDA receptors is believed responsible for the generation of “neuropathic” pain, a type of pain which is sometimes called “neurogenic pain” or “wind-up” pain (Woolf et al 1989; Kristensen et al 1992; Yamamoto and Yaksh 1992).

By mechanisms that are poorly understood, pathological changes associated with diabetes are conducive to the generation of neuropathic pain, a condition known as “diabetic neuropathy”. One of the distinguishing characteristics of neuropathic pain is that morphine and related pain-killing drugs which are effective in controlling other types of pain are usually ineffective in controlling neuropathic pain (Backonja 1994). NMDA receptors are found in the pain-transmitting structures of the spinal cord, thalamus and certain layers of the cerebral cortex and several recent reports indicate that NMDA antagonists can prevent or ameliorate neuropathic pain (Davar et al 1991; Mao et al 1992; Seltzer et al 1991; Neugebauer et al 1993; Kristensen et al 1992; Backonja et al 1994).

For example, intractable tic douloureux (Chesire, W. P., Clin. J. Pain, 11: 139-142, 1995), has been effectively treated with felbamate at doses of 1200-2400 mg/day. Conditions such as peripheral neuropathy, terminal cancer pain and failed back surgery which are intractable to current treatment modalities also benefit from felbamate treatment. In the formalin injection animal pain model, glycine site antagonists (Millan, M. J., Neurosci. Lett., 178(1):139-143, 1994, Vaccarino, A. L., Brain Res., 615(2):331-334, 1993) decreased the late phase pain response. In a rat model of painful peripheral neuropathy, felbamate produced significant reductions in all measures of pain (Imamura, I., J. Pharm. Exp. The., 275(1):177-182, 1995). Specifically, the action of felbamate was antihyperalgesic and antiallodynic rather than analgesic.

The NMDA receptor antagonist felbamate derivatives of the present invention function similar to felbamate to block the NMDA receptor at the glycine site and thus these compounds will also prevent chronic pain transmission. Therefore, in accordance with one embodiment, the felbamate derivatives of the present invention are utilized to provide symptomatic relief from neuropathic pain without producing central nervous system side effects.

In accordance with one embodiment, the felbamate derivatives are administered chronically in oral doses ranging from 100-15,000 mg/day, and more preferable about 1200 to about 7200 mg/day (serum levels ranging from about 25 ug/ml to about 300 ug/ml), to alleviating chronic pain.

It has been reported that felbamate is effective in treating the following conditions/diseases associated with excessive activation of the NMDA receptor: Sepsis, Meningitis, CNS Vasculitis, Adrenoleukodystrophy, Impotence, Schizophrenia, Drug Addiction, Multiple Sclerosis, Fatigue (including fatigue associated with chronic diseases, such as multiple sclerosis, post-polio syndrome and Parkinson's, as well as chronic fatigue syndrome), Lead Poisoning, Mitochondrial Myopathies, HIV Dementia, Depression, Amyotrophic Lateral Sclerosis, Parkinson's Disease, Attention Deficit Disorder, Narcolepsy, Alzheimer's Disease, Childbirth Complications (i.e. premature labor, prolonged labor, hypoxia, etc. which place the fetus at risk for cerebral ischemic damage and cerebral palsy), Surgical Anesthesia (as a prophylactic treatment to reduce risk of brain damage from hypoxia, anoxia, cerebral embolism (i.e., fat, air), hypotension, hypoglycemia etc.), Traumatic Head and Spinal Cord Injury, Hypoglycemia, Tourette's Syndrome and Hepatic Encephalopathy, see U.S. Pat. No. 5,728,728, the disclosure of which is incorporated herein. Accordingly, it is anticipated that the NMDA receptor antagonists of the present invention can be used to treat these and other conditions that are associated with excessive activation of the NMDA receptor. In particular, it has been reported that NMDA receptor antagonists have efficacy in treating glaucoma patients that do not respond to standard therapy. Thus in accordance with one embodiment, the felbamate derivatives of the present invention are used in a method for treating a patient suffering from glaucoma or other conditions associated with excessive activation of the NMDA receptor. The method comprises the step of administering an effective amount of the felbamate derivative.

In one embodiment of the present invention a pharmaceutical composition comprising a felbamate derivative of the present invention is administered to a patient suffering from Alzheimer's Disease (DAT) to alleviate symptoms associated with the disease. Alzheimer's Disease (DAT) is a progressive dementing illness which is caused by an abnormal form of amyloid deposition in the brain. Excessive amyloid deposition induces glutamate toxicity of the NMDA receptor, resulting in neuronal death in areas of the brain that have a high density of NMDA receptors such as the hippocampus and cerebral cortex (areas of maximal neuronal death in DAT). The decrease in binding of NMDA receptors in DAT visual cortex is correlated with increased numbers of neurofibrillary tangles. (Carlson, M. D., Neurobiol. Aging, 14(4): 343-352, 1993). Studies in animals have shown that glycine antagonist improve learning and attenuate scopalamine memory deficits (Fishkin, R. J., Behav. Neurol. Biol., 59(2):150-157, 1993, Finkelstein, J. E., Pharmacol. Biochem. Behav., 49(3):707-710, 1994, Baxter, M. G., Neurobiol. Aging, 15(2):207-213, 1994). Accordingly, the felbamate derivatives of the present invention, having glycine site NMDA antagonist with cognitive enhancement and neuronal protection properties, are useful for treating DAT and preventing DAT associated neuronal degeneration.

In another embodiment of the present invention a pharmaceutical composition comprising a felbamate derivative of the present invention is administered to treat a patient suffering from Parkinson's Disease. Parkinson's Disease (PD) is a selective degeneration of predominately D₂ dopaminergic neurons in the substantia nigra (motor portion of the basal ganglia) which produces progressive motor symptoms of rigidity and bradykinesia (slowness of movement). An NMDA-excitatory mechanism of early neuronal cell death is involved in the etiology of PD. Felbamate has been shown to antagonize the D₂ (dopamine receptor) in an animal model of cataplexy (Kretchnmer, B. D., Neurosci Lett., 179(1-2):115-118, 1994). Accordingly, the felbamate derivatives of the present invention, having NMDA glycine site antagonist activity prevents NMDA receptors from being excessively stimulated, thus preventing progressive motor weakness and death (neuroprotection) in PD.

In another embodiment of the present invention a pharmaceutical composition comprising a felbamate derivative of the present invention is administered to treat a patient suffering from HIV Dementia. Patients who become HIV(+) have early pre-clinical brain neuronal deterioration as measured by NMR spectroscopy. When HIV(+) patients convert to AIDS, brain involvement produces dementia and is universally fatal. The mechanisms of brain deterioration appear to involve production of substances (i.e., quinolinic acid) that activate NMDA receptors and cause neuronal death by inducing intra cellular Ca.sup.++ overload. Accordingly, the felbamate derivatives of the present invention, having NMDA glycine site antagonist activity function as neuroprotection agents and prevent neuronal death.

When administered orally, the compounds of the present invention can be administered as a liquid solution, powder, tablet, capsule or lozenge. The felbamate derivative compounds can be used in combination with one or more conventional pharmaceutical additive or excipients used in the preparation of tablets, capsules, lozenges and other orally administrable forms. When administered as an intravenous solution, the derivatives of the present invention can be admixed with conventional IV solutions.

The compounds of the present invention are administered at a dose range effective to alleviate the symptoms associated with the disorder. In accordance with one embodiment the felbamate derivative (active agent) is administered in a dosage form of about 0.1 mg/kg to about 5.0 g/kg, and more particularly about 0.25 mg/kg to about 1 g/kg. The dosage will vary based on the route of administration and the condition/disorder to be treated.

### EXAMPLE 1

Use of an LC/MS Method to Quantify the two Atropaldehyde Derived Mercaptuic Aids 7 and 8 in a Patient Population Treated with Felbamate.

Although the FDA has recommended patients be given felbamate therapy only when other therapies have failed, it is estimated that 8,000-12,000 patients remain on felbamate therapy in the United States (12). While the mercapturic acids are generated from addition to glutathione, any similar nucleophile (i.e., nucleophilic amino acids of proteins or DNA bases) would be expected to undergo conjugation to atropaldehyde. The more atropaldehyde generated in vivo the greater the likelihood that a critical target will be alkylated, leading to toxicity. Therefore, it is reasonable to expect that the potential for toxicity will correlate to the amount of atropaldehyde formed. Further, the amount of atropaldehyde derived mercapturic acids excreted in urine will be a function of the amount of atropaldehyde formed.

In accordance with one aspect of the present invention an analytical method is described for quantifying the relevant metabolites excreted in patient urine as a measurement of a patient's susceptibility to toxicity associated with felbamate therapy. The method of determining a patient's susceptibility to adverse side affects from felbamate administration comprises the steps of obtaining a biological sample from the patient, preferably a urine sample, quantifying the mercapturic acid metabolites present in the sample and extrapolating the amount of atropaldehyde formed. In accordance with one embodiment standard liquid chromatography and mass spectroscopy (LC/MS) is used for quantifying the relevant metabolites excreted in patient urine.

Materials and Methods

Chemicals and Instruments. All reagents were purchased from either Aldrich Chemical Co. or Sigma Chemical Co. and were of the highest quality available. HPLC was performed on a Waters 2690 Separations Module with a Waters 484 tunable absorbance detector (at 214 nm) using a Waters Symmetry C₁₈ (2.1 mm×150 mm) column. Mass Spectra were obtained by coupling this LC system to a Finnigan MAT LCQ ion trap mass spectrometer equipped with an electrospray ionization source. NMR spectra were recorded on a General Electric QE300 spectrometer at 300 MHz and chemical shifts are reported in ppm. Melting points were determined on a Thomas-Hoover UNI-MELT apparatus and are uncorrected.

Synthesis. 2-Phenyl-(1,1,3,3-tetra-deuterio)-1,3-propanediol. 2-Phenyl-(1,1,3,3-tetra-deuterio)-1,3-propanediol was obtained by reduction of diethyl phenylmalonate with LiA1D₄ using methodology described previously for the formation of 2-phenyl-1-1,3-propanediol (1). The isotopic purity of the product was determined to be ≧98% as assessed by ¹H NMR and GC/MS. (mp=48-50° C.) ¹H NMR (CDCl₃): δ2.6 (s, 2H), 3.0 (s, 1H), 7.3 (m, 5H). ¹³C NMR (CDCl₃): δ49.8, 127.7, 128.5, 129.3, 139.9. GC/MS (CI-methane)MH⁺=157—fragment ions: 139, 121, 106, 93. Elemental Analysis: theoretical C, 69.20; H, 7.74 found C, 68.98; H, 7.68. Molecular mass is calculated with 4²H atoms but the instrument used for elemental analysis observes each deuterium as though it were hydrogen. Therefore, the theoretical value for elemental analysis of hydrogen is determined based on the presence of 12 ¹H (i.e., 12×1.008=12.096/156.21=7.74%).

2-Phenyl-(I,I,3,3-tetra-deuterio)-1,3-propanediol monocarbamate. The d₄-monocarbamate alcohol was prepared from the d₄-diol using methodology described previously (1). The isotopic purity of the product was determined to be ≧98% as assessed by ¹H NMR and LC/MS. (mp=71-72° C.) ¹H NMR ((CD₃)₂CO): δ3.1 (s,1H), 4.8 (bs, 2H), 7.3 (m, 5H). LC/ESI-MS: MH^{×}=200.0.

3-Carbamoyl-(3,3-di-deuterio)-2-phenylpropionic acid. The d₂-acid carbamate was obtained essentially as described by Adusumalli et al. except that the d₄-monocarbamate

alcohol was used as starting material (10). The isotopic purity of the product was determined to be ≧98% as assessed by ¹H NMR and LC/MS. (mp=99-102° C.) ¹H NMR ((CD₃)₂SO): δ 3.9 (s, 1H), 5.8 (bs, 2H), 7.3 (m, 5H), 12.4 (bs, 1H). ¹³C NMR ((CD₃)₂C0): δ54.1, 127.3, 128.7, 128.7, 139.3, 155.6, 201. 7. LC/ESI-MS: MH⁺=211.9. Elemental Analysis: theoretical C, 56.87; H, 5.25; N, 6.63 found C, 56.74; H, 5.31; N, 6.57.

2-Phenyl-(1,1,3,3-tetra-deuterio)-1,3-propanediol dicarbamate. d₄-Felbamate was made using methodology described previously except that d₄-monocarbamate alcohol was used as starting material (2). The isotopic purity of the product was determined to be ≧98% as assessed by ¹H NMR and LC/MS. The spectral data obtained for this compound are in agreement with published values (11). (mp=148-150 C) ¹H NMR ((CD₃)₂S0): δ 3.1 (s, 1H), 6.4 (bs, 4H), 7.2 (m, 5H). LC/ESI-MS: MH⁺=243.1. Elemental Analysis: theoretical C, 54.53; H, 5.83; N, 11.56 found C, 54.63; H, 5.84; N, 11.64.

N-d₃-acetyl-L-cysteine. Acetic anhydride (d₆) (0.29 g, 2.7 mmol) was added to a solution of Cys(trt)-OH in 20 mL DMF and 1 mL pyridine and stirred overnight. The reaction mixture was diluted with 50 mL ether and extracted with saturated aqueous lithium bromide (2×50 mL). The organics were dried over sodium sulfate and the solvent was removed under reduced pressure. This intermediate was purified by flash chromatography using 1:1 methanol and chloroform affording a white solid: ¹H NMR (CDCI₃) δ 7.37-7.12 (m, 15H), 4.13 (m, 1H), 2.60 (m, 2H). The sulfide was deprotected using a solution of TFA in dichloromethane (1:1) for 2 hr. The solvents were removed under reduced pressure and the crude product was used in the next reaction.

N-d₃-acetyl-S-(2-phenylpropan-3-ol)-L-cysteine. The d₃-Nac-alcohol was formed using methodology described previously except that N-d₃-acetyl-cysteine was used in the formation of the d₃-Nac-atropaldehyde intermediate (2). The isotopic purity of the product was determined to be≧95% as assessed by ¹H NMR and LC/MS. ¹H NMR (D₂0): δ 2.66-3.0 (m, 6H), 3.74 (t, 1H, J=5.4 Hz), 4.4 (m, 1H), 7.3 (m, 5H). LC/MS: MH⁺=301.3.

N-d₃-acetyl-S-(2-phenylpropanoic acid)-L-cysteine. The d₃-Nac-acid was formed using methodology described previously except that N-d₃-acetyl-cysteine was used in the reaction with 2-phenyl acrylic acid (2). The isotopic purity of the product was determined to be ≧95% as assessed by ¹H NMR and LC/MS. ¹H NMR (D₂0): δ 2.75-3.23 (m, 4H), 3.82 (t,1H, J=5.5 Hz), 4.4 (m, 1H), 7.32 (m, 5H). LC/ESI-MS: MH⁺=315.2.

Preparation of Patient Urine Samples. Urine samples were obtained from patient volunteers undergoing felbamate therapy for control of epileptic seizures and under the care of physicians at either the University of Virginia (Charlottesville, Va.) or the EpiCare Center (Memphis, Tenn.). Urine samples were diluted four fold with distilled water (this was done prior to overnight shipment for samples requiring shipping) and placed in an orbital shaking water bath for˜20 min at 37° C. to insure that all of the analytes were in solution. 500 μL of this diluted sample were removed and added to 100 μL of a mixture of the four deuterated internal standards. The concentration of standards in the mixture resulted in the addition of 563 nmol d₄-felbamate, 140 nmol d₂-acid carbamate, 54.0 nmol d₃-Nac-alcohol, and 27.5 nmol d₃-Nac-acid to each 500 μL diluted urine sample. After mixing, 200 μL were removed and added to 20 μL of 20% H0Ac. This acidified sample was then applied to a preconditioned Waters “Oasis” solid phase extraction cartridge (Waters Corp., Woburn, Mass.). The cartridge was washed with 2 mL 0.1% HOAc followed by 3 mL 10% CH₃CN/90% water (0.1%) HOAc. The analytes and internal standards were then eluted with 3 mL 30% CH₃CN:70% (0.1%) HOAc. This fraction was then analyzed by LC/MS without further manipulation.

LC/MS Analysis of Urine Samples for Metabolite Quantification. LC/MS analysis was performed using a Waters 2690 HPLC equipped with an autosampler and a Waters 486 tunable absorbance detector. This LC system was interfaced to a Finnigan MAT LCQ ion trap mass spectrometer. A 10 μL injection of the fraction from solid phase extraction was applied to a Waters Symmetry C₈ reversed phase column (33% CH₃CN:67% (0.1%) H0Ac, 2.1 mm×150 mm, 0.2 mL/min). The post-column flow was directed through a Waters 486 tunable absorbance detector (10 μL flow cell, λ=214 nm) which was used for qualitative assessment of the sample and not for quantitation. The flow was then directed to the electrospray ionization source of the LCQ.

The mass spectrometer was programmed to collect data in full scan mode from 190-320 m/z and was tuned to maximize the signal from the analytes under the HPLC conditions. The values for the electrospray parameters were as follows: heated capillary temperature=180° C.; spray voltage=5.6 kV; capillary voltage=20 V; sheath gas (nitrogen) flow rate=70; auxiliary gas (helium) flow rate=20. The automatic gain control (AGC) was on with a target ion count of 7×10⁷ ions and a maximum ion inject time of 150 ms. The scan time was˜0.5 s. Linear response curves were observed for each analyte and internal standard pair over approximately two orders of magnitude range centered on the absolute amount of each internal standard added to the samples. The amount of analytes in the patient urine samples fell within these linear response ranges.

Quantification was achieved by integration of the peaks from the mass chromatogram for each analyte using software (Navigator 1.1) provided with the LCQ. The area (expressed as counts×seconds) of the peak for each metabolite was compared to the area for its corresponding deuterated internal standard. As the absolute amount of internal standard added per mL of urine was known, the absolute amount of analyte per mL of urine could be determined.

Results

Analysis was performed on 34 urine samples from 31 patients undergoing felbamate therapy for control of epileptic seizures. As is common for epileptics, many of these patients (n=19) were undergoing polytherapy for seizure control with the remainder (n=12) undergoing felbamate monotherapy. The age of this patient pool (14 men and 17 women) spanned from 10-57 years old with a mean age of 37. All of the urine samples analyzed were found to contain both of the mercapturic acids, indicating that formation of atropaldehyde in vivo does occur in the patient population and appears to be universal. More Nac-alcohol 7 was excreted than Nac-acid 8 for all of the patients. The average ratio of Nac-alcohol to Nac-acid was 6.4, however the values varied widely (ratios=2-14).

The absolute amounts of the analytes excreted per mL of urine varied considerably between patients. For example, the amount of felbamate excreted ranged from 819±8.5 nMoles/mL (this patient had a total daily dose of 3.6 grams of felbamate) to 10,064±515 nMoles/mL (this patient had a total daily dose of 6.0 grams of felbamate). This illustrates the effect of urine volume on the results obtained from this method. Though the dosage difference was less than double, the difference in amount of felbamate excreted per mL was greater than ten times. Thus, to compare the results from one patient to another, the values for the metabolites were normalized to the amount of felbamate.

Discussion

We have developed an isotope-dilution based LC/MS method to quantify the amounts of felbamate 1, the acid carbamate 4, and the two mercapturic acids 7 and 8 excreted in patient urine. Although the FDA has recommended patients be given felbamate therapy when other therapies have failed, it is estimated that 8,000-12,000 patients remain on felbamate therapy in the United States (12).

Scheme I illustrates the metabolic pathway leading to atropaldehyde formation and its disposition. The aldehyde carbamate represents the “commitment” step. It is at this point that a molecule is either committed to the toxic pathway of atropaldehyde 5 or the detoxification pathway of the acid carbamate 4. The amount of mercapturic acids 7 and 8 excreted in urine will mirror the flux through the “toxic” pathway. That is, an individual that generates high levels of atropaldehyde would be expected to have correspondingly high levels of the mercapturic acids. Therefore, the ratio of acid carbamate excreted compared to the combined mercapturic acids would describe the disposition of the aldehyde carbamate for a given patient. The values for the two mercapturic acids can be combined as they both represent the same pathway of aldehyde carbamate disposition. Of course, there are other factors that may modulate the disposition through these two pathways (i.e., co-administration of modulators of enzyme activity or glutathione levels), but this appears to be a promising approach to evaluating the metabolic distribution between toxic and non-toxic pathways in a patient population.

We applied this LC/MS method to the analysis of 34 urine samples from 31 patients undergoing felbamate therapy for control of epileptic seizures. All of these patients have been undergoing felbamate therapy for several years, without any of the severe side effects. As the severe toxicities associated with felbamate demonstrate a mean onset time of ≦6 months, these patients probably constitute a population of “normal” or “safe” metabolizers of felbamate.

The data obtained from the urine samples is illustrated that there is a “normal” range for the distribution of the aldehyde carbamate between the acid carbamate and atropaldehyde (as represented by the mercapturic acids). There does not appear to be a significant correlation between sex or therapy type and the relative amounts of metabolites formed. An individual with high esterase activity would produce relatively more of the monocarbamate alcohol from felbamate leading to increased generation of all subsequent metabolites. However, the ratio of the metabolites may still be very similar.

By our hypothesis, alkylation of proteins by atropaldehyde may result in the generation of antigens that precipitate an immune response in a manner similar to mechanisms of immune mediated toxicity for other agents. If the toxicity due to atropaldehyde is immune mediated, then susceptibility to this toxicity will be a function not only of the formation of atropaldehyde-protein conjugates but also of a patient's immune system phenotype. Some patients may have a particular phenotype that makes them allergic to atropaldehyde-protein conjugates while others do not exhibit this response. Alternatively, everyone may have the potential for an immune response, but the amount of atropaldehyde protein conjugates produced must reach a critical level before the immune response occurs. That is, all of the patients may be producing low levels of antigens, but the levels are not normally high enough to trigger an immune response. It is not until a secondary event, such as inhibition of acid carbamate formation or glutathione depletion, occurs that the production of antigens surpasses the critical level and toxicity is manifest.

Because of the potential for an immune mediated component for felbamate toxicity by our hypothesis, it may be important to understand both an individual's level of atropaldehyde formation and their phenotype to develop a complete screening method. The method described above has demonstrated sufficient precision for the identification of “outliers” and appears to hold potential for the monitoring of patients undergoing felbamate therapy.

### EXAMPLE 2

A General Synthesis of 2-Substituted-2-fluoro-1,3-propanediol Dicarbamate and Monocarbamate Derivatives

Materials and Methods

Chemicals and Instruments. All reagents were purchased from Aldrich Chemical Co. and were of the highest quality available. HPLC was performed on a Waters 2690 Separations Module with a Waters 484 tunable absorbance detector (at 214 nm) using a Waters Symmetry C18 (2.1 mm×150 mm) column. Mass Spectra were obtained by coupling this LC system to a Finnigan MAT LCQ ion trap mass spectrometer equipped with an electrospray ionization source. NMR spectra were recorded on a General Electric QE300 spectrometer at 300 MHz and chemical shifts are reported in ppm. Melting points were determined on a Thomas-Hoover UNI-MELT apparatus and are uncorrected.

The 2-substituted-2-fluoro-1,3-propanediol dicarbamate and monocarbamate derivatives were synthesized through the general sequence outlined below. We have found this sequence to be amenable to a wide range of substitutents at the 2-position of the 1,3-propane diol skeleton and to provide excellent yields of finished product. Briefly, an appropriately substituted diethyl malonate species was fluorinated at the methine position using sodium hydride as base and Selectfluor® as the fluorinating reagent. The resultant diethyl 2-substituted-2-fluoromalonate was then reduced to the corresponding 2-substituted-2-fluoro-1,3-propanediol with lithium aluminum hydride at low temperature. The 2-substituted-2-fluoro-1,3-propanediol species is then treated with either a slight excess of carbamoylation reagent (1.1 equivalents) or 2.25 equivalents of carbamoylation reagent to produce predominantly the monocarbamate derivative or dicarbamate derivative, respectively. A preferred embodiment of this sequence describing the syntheses of 2-fluoro-2-(2′-thienyl)-1,3,-propanediol monocarbamate and dicarbamate is provided below.

General Scheme

Diethyl 2-fluoro-2-(2′-thienyl)-malonate. The title compound was obtained by the method described by Lal.¹ By this method, diethyl 2-(2′-thienyl)malonate (10.0 g, 44.3 mmol) was slowly added to a solution of NaH (1.22 g, 1.2 equiv.), in ethyl ether or THF (420 mL). After 30 min, Selectfluor®(16.5 g, 1.1 equiv.) was added and the solution was stirred for 12 hours at room temperature. The solution was filtered through a 40 micron glass fritted filter and concentrated to afford the title compound (10.1 g, 40.8 mmole) in 92% yield as an oil.

2-Fluoro-2-(2′-thienyl)-1,3-propanediol. 2-Fluoro-2-(2′-thienyl)-1,3-propanediol was obtained from diethyl 2-fluoro-2-(2′-thienyl)malonate by lithium aluminum hydride reduction using methodology analogous to that described previously for the formation of 2-phenyl-1,3-propanediol.² However, the reduction was initiated at −40° C. and was allowed to warm to room temperature over one hour, followed by stirring for an additional 1 to 2 hours, at which time the reaction was complete by TLC. By this method, diethyl 2-fluoro-2-(2′-thienyl)-malonate (10.1 g, 40.8 mmole) in a solution of ethyl ether (100 mL) was added to a slurry of lithium aluminm hydride (180 mg, 5.0 mmole) in ethyl ether (250 mL) at −40° C. The reaction was stirred for an hour and subsequently allowed to warm to room temperature and then stirred for an additional 2 hours. The reaction was then cooled to −78° C. then quenched with water (200 μL), then 10% sodium hydroxide solution (200 μL), followed by water (600 μL). The solution was allowed to warm to 0° C. over 1 hour, then filtered. Concentration of the solution and silica gel chromatography with ethyl ether elution afforded the title compound (4.40 g, 30.6 mmole) in 75% yield.

2-Fluoro-2-(2′-thienyl)-1, 3-propanediol dicarbamate. The title compound (IUPAC: 3-(aminocarbonyloxy)-2-fluoro-2-(2′-thienyl)propyl aminooate) was made from 2-fluoro-2-(2′-thienyl)-l, 3-propanediol as follows. This procedure represents a modification of the method of Adusumalli et al³ for synthesis of felbamate. To a solution of 2-fluoro-2-(2′-thienyl)-1,3-propanediol (4.40 g, 30.6 mmole) in tetrahydrofuran (100 mL) was added 1,1′-carbonyldiimidazole (12.3 g, 76 mmole) at room temperature. After 4 hours at room temperature, the reaction mixture was cooled to −78° C. and ammonia was bubbled in for 20 minutes. The resulting mixture was stirred overnight, concentrated under rotary evaporation, filtered and chromatographed on silica gel using ethyl ether eluent to provide the title compound as a pristine white solid (4.82 g, 18.4 mmole) in 60% yield.

2-Fluoro-2-(2′-thienyl)-1, 3-propanediol monocarbamate. To a slurry of sodium hydride (520 mg, 21.6 mmole) in tetrahydrofuran (50 mL) was added 2-fluoro-2-(2′-thienyl)-1,3-propanediol (2.88 g, 20 mmole) in tetrahydrofuran (50 mL) at room temperature. After 2 hours, t-butyldimethylsilyl chloride (1.50 g, 19.5 mmole) was added in one portion and the resulting mixture was allowed to stir until complete by thin layer chromatography (30-60 minutes). 1,1 ′Carbonyldiimidazole (3.50 g, 21.6 mmole) was added to the mixture in one portion. After 2 hours, the mixture was cooled to −78° C. and ammonia ws bubbled into the vessel for 10 minutes. The resulting mixture was stirred overnight and concetrated under rotary evaporation. The concentrate was diluted with 30 mL of methanol and aqueous hydrochloric acid was added (10 mL of a 2.5N solution) providing a clear solution that was viorously stirred for another hour. The reaction mixture was concetrated under rotary evaporation, diluted with water and extracted 3 times with 10 mL aliquots of ethyl ether. The organic extracts were combined and concetrated by rotary evaporation. The product was purified by gradient chromatogarphy using an initial eluent of ether/petroleum ether (1:1) which was raised to 100% ethyl ether. The product was obtained as a white crystalline material in 67% (2.49 g, 1.33 mmole).

1. A compound having the general structure 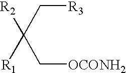
wherein R₂ is F or Cl;
R₃ is hydroxy or —OCONH₂;
R₁ is selected from the group consisting of C₁-C₉ alkyl, C₃-C₉ cycloalkyl, C₁-C₉ alkylated C₃-C₉ cycloalkyl, 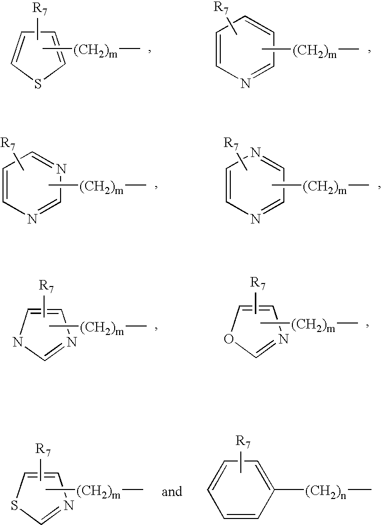
m is 0-3, n is 1-3 and R₇ is selected from the group consisting of H, halo, alkyl, haloalkyl and hydroxy.

2. The compound of claim 1 wherein R₂ is F.

3. The compound of claim 2 wherein R₇ is H and R₃ is —OCONH₂.

4. The compound of claim 2 wherein R₁ is selected from the group consisting of C₁-C₉ alkyl, C₃-C₉ cycloalkyl, C₁-C₉ alkylated C₃-C₉ cycloalkyl, 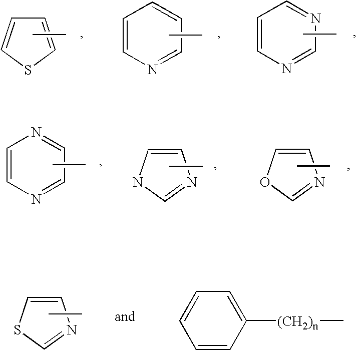
wherein n is 1-3 and R₃ is —OCONH₂.

5. A compound having the general structure 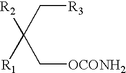
wherein R₁ is selected from the group consisting of C₃-C₉ cycloalkyl, 2-thienyl, 3-thienyl, 2-pyridinyl, 3-pyridinyl, 4-pyridinyl, 2-(1,3 diazinyl), 4-(1,3 diazinyl), 5-(1,3 diazinyl), 2-(1, 4 diazinyl), 2-imidazoyl, 4-imidazoyl, 2-(1, 3 oxazinyl), 4-(1, 3 oxazinyl), 5-(1, 3 oxazinyl), 2-(thiazinyl), 4-(thiazinyl), 5-(thiazinyl) and 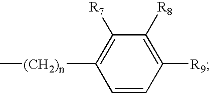
n is 1-3;
R₂ is F or Cl;
R₃ is hydroxy or —OCONH₂; and
R₇, R₈ and R₉ are independently selected from the group consisting of H, halo, alkyl, haloalkyl and hydroxy.

6. The compound of claim 5 wherein R₂ is F; and
R₃ is —OCONH₂.

7. A method for treating a patient suffering from neuropathic pain, said method comprising the step of administering a composition comprising a compound having the general structure 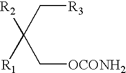
wherein R₂ is F or Cl;
R₃ is hydroxy or —OCONH₂;
R₁ is selected from the group consisting of C₁-C₉ alkyl, C₃-C₉ cycloalkyl, C₁-C₉ alkylated C₃-C₉ cycloalkyl, 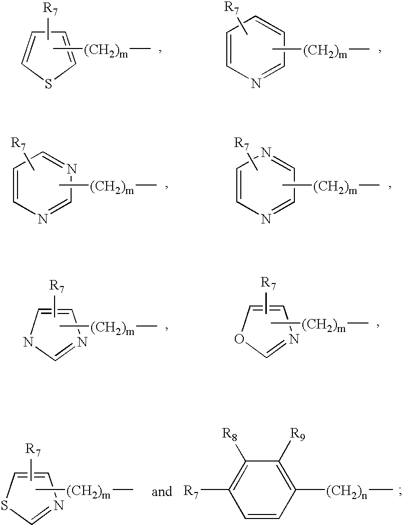
m is 0-3, n is 1-3; and
R₇, R₈ and R₉ are independently selected from the group consisting of H, halo, alkyl, haloalkyl and hydroxy.

8. The method of claim 7 wherein R₂ is F.

9. The method of claim 8 wherein R₃ is —OCONH₂.

10. The method of claim 9 wherein R₇, R₈ and R₉ are each H.

11. The method of claim 9 wherein R₁ is selected from the group consisting of C₃-C₉ cycloalkyl, 2-thienyl, 3-thienyl, 2-pyridinyl, 3-pyridinyl, 4-pyridinyl, 2-(1,3 diazinyl), 4-(1,3 diazinyl), 5-(1,3 diazinyl), 2-(1, 4 diazinyl), 2-imidazoyl, 4-imidazoyl, 2-(1, 3 oxazinyl), 4-(1, 3 oxazinyl), 5-(1, 3 oxazinyl), 2-(thiazinyl), 4-(thiazinyl), 5-(thiazinyl) and

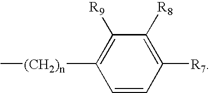

12. The method of claim 7 wherein the composition is administered orally.

13. The method of claim 12 wherein the unit dosage form of the composition comprises about 0.1 mg/kg to about 1 g/kg of said compound.

14. A method for treating a patient suffering from a neurological disorder, said method comprising the step of administering a composition comprising a compound having the general structure 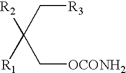
wherein R₂ is F or Cl;
R₃ is hydroxy or —OCONH₂;
R₁ is selected from the group consisting of C₁-C₉ alkyl, C₃-C₉ cycloalkyl, C₁-C₉ alkylated C₃-C₉ cycloalkyl, 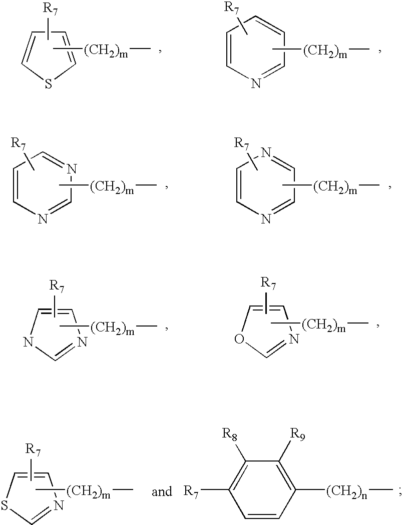
m is 0-3,n is 1-3; and
R₇, R₈ and R₉ are independently selected from the group consisting of H, halo, alkyl, haloalkyl and hydroxy.

15. The method of claim 14 wherein R₂ is F.

16. The method of claim 15 wherein R₃ is —OCONH₂.

17. The method of claim 16 wherein R₇, R₈ and R₉ are each H.

18. The method of claim 16 wherein R₁ is selected from the group consisting of C₃-C₉ cycloalkyl, 2-thienyl, 3-thienyl, 2-pyridinyl, 3-pyridinyl, 4 -pyridinyl, 2-(1,3 diazinyl), 4-(1,3 diazinyl), 5-(1,3 diazinyl), 2-(1, 4 diazinyl), 2-imidazoyl, 4-imidazoyl, 2-(1, 3 oxazinyl), 4-(1, 3 oxazinyl), 5-(1, 3 oxazinyl), 2-(thiazinyl), 4-(thiazinyl), 5-(thiazinyl) and

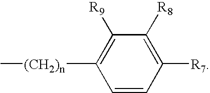

19. The method of claim 14 wherein the composition is administered orally.

20. The method of claim 19 wherein the unit dosage form of the composition comprises about 0.1 mg/kg to about 1 g/kg of said compound.

21. A method for treating a patient suffering from tissue damage resulting from localized hypoxic conditions, said method comprising the step of administering a composition comprising a compound having the general structure 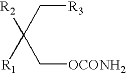
wherein R₂ is F or Cl;
R₃ is hydroxy or —OCONH₂;
R₁ is selected from the group consisting of C₁-C₉ alkyl, C₃-C₉ cycloalkyl, C₁-C₉ alkylated C₃-C₉ cycloalkyl, 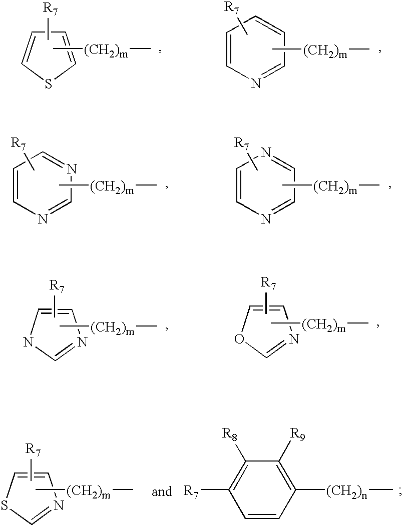
m is 0-3, n is 1-3; and
R₇, R₈ and R₉ are independently selected from the group consisting of H, halo, alkyl, haloalkyl and hydroxy.

22. The method of claim 21 wherein R₂ is F.

23. The method of claim 22 wherein R₃ is —OCONH₂.

24. The method of claim 23 wherein R₇, R₈ and R₉ are each H.

25. The method of claim 23 wherein R₁ is selected from the group consisting of C₃-C9 cycloalkyl, 2-thienyl, 3-thienyl, 2-pyridinyl, 3-pyridinyl, 4-pyridinyl, 2-(1,3 diazinyl), 4-(1,3 diazinyl), 5-(1,3 diazinyl), 2-(1, 4 diazinyl), 2-imidazoyl, 4-imidazoyl, 2-(1, 3 oxazinyl), 4-(1, 3 oxazinyl), 5-(1, 3 oxazinyl), 2-(thiazinyl), 4-(thiazinyl), 5-(thiazinyl) and

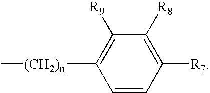

26. The method of claim 21 wherein the composition is administered orally.

27. The method of claim 26 wherein the unit dosage form of the composition comprises about 0.1 mg/kg to about 1 g/kg of said compound.

28. A method for treating glaucoma, said method comprising the step of administering a composition comprising a compound having the general structure 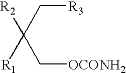
wherein R₂ is F or Cl;
R₃ is hydroxy or —OCONH₂;
R₁ is selected from the group consisting of C₁-C₉ alkyl, C₃-C₉ cycloalkyl, C₁-C₉ alkylated C₃-C₉ cycloalkyl, 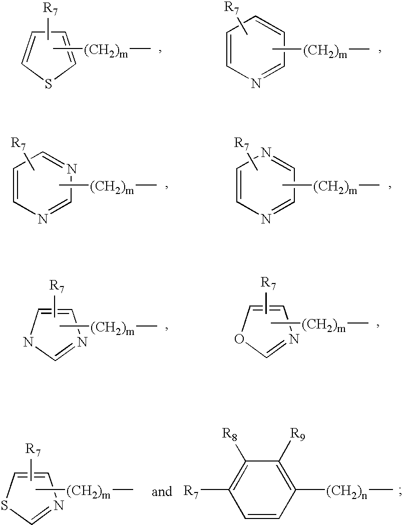
m is 0-3,n is 1-3; and
R₇, R₈ and R₉ are independently selected from the group consisting of H, halo, alkyl, haloalkyl and hydroxy.

29. The method of claim 28 wherein R₂ is F.

30. The method of claim 29 wherein R₃ is —OCONH₂.

31. The method of claim 30 wherein R₇, R₈ and R₉ are each H.

32. The method of claim 30 wherein R₁ is selected from the group consisting of C₃-C₉ cycloalkyl, 2-thienyl, 3-thienyl, 2-pyridinyl, 3-pyridinyl, 4-pyridinyl, 2-(1,3 diazinyl), 4-(1,3 diazinyl), 5-(1,3 diazinyl), 2-(1, 4 diazinyl), 2-imidazoyl, 4-imidazoyl, 2-(1, 3 oxazinyl), 4-(1, 3 oxazinyl), 5-(1, 3 oxazinyl), 2-(thiazinyl), 4-(thiazinyl), 5-(thiazinyl) and

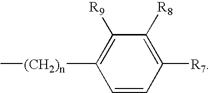

33. The method of claim 28 wherein the composition is administered orally.

34. The method of claim 33 wherein the unit dosage form of the composition comprises about 0.1 mg/kg to about 1 g/kg of said compound.

35. A pharmaceutical composition comprising a compound having the general formula 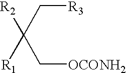
wherein R₂ is F or Cl;
R₃ is hydroxy or —OCONH₂;
R₁ is selected from the group consisting of C₁-C₉ alkyl, C₃-C₉ cycloalkyl, C₁-C₉ alkylated C₃-C₉ cycloalkyl, 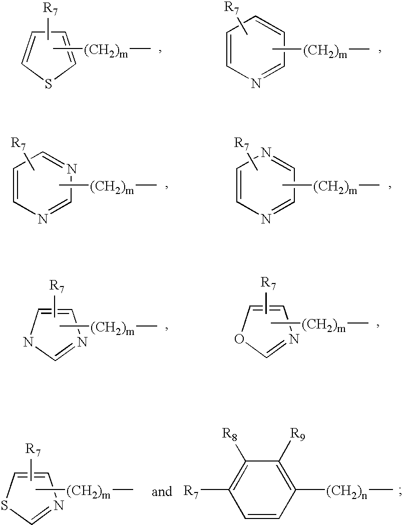
m is 0-3,n is 1-3; and
R₇, R₈ and R₉ are independently selected from the group consisting of H, halo, alkyl, haloalkyl and hydroxy.

36. The pharmaceutical composition of claim 35 wherein R₂ is F.

37. The pharmaceutical composition of claim 36 wherein R₃ is —OCONH₂.

38. The pharmaceutical composition of claim 37 wherein R₇, R₈ and R₉ are each H.

39. The pharmaceutical composition of claim 37 wherein R₁ is selected from the group consisting of C₃-C₉ cycloalkyl, 2-thienyl, 3-thienyl, 2-pyridinyl, 3-pyridinyl, 4-pyridinyl, 2-(1,3 diazinyl), 4-(1,3 diazinyl), 5-(1,3 diazinyl), 2-(1, 4 diazinyl), 2-imidazoyl, 4-imidazoyl, 2-(1, 3 oxazinyl), 4-(1, 3 oxazinyl), 5-(1, 3 oxazinyl), 2-(thiazinyl), 4-(thiazinyl), 5-(thiazinyl) and

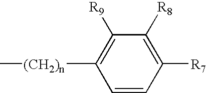

40. A pharmaceutical composition comprising a compound having the general formula 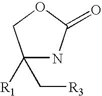
wherein R₁ is selected from the group consisting of C₁-C₉ alkyl, C₃-C₉ cycloalkyl, C₁-C₉ alkylated C₃-C₉ cycloalkyl, C₃-C₉ cycloalkyl, 2-thienyl, 3-thienyl, 2-pyridinyl, 3-pyridinyl, 4-pyridinyl, 2-(1,3 diazinyl), 4-(1,3 diazinyl), 5-(1,3 diazinyl), 2-(1, 4 diazinyl), 2-imidazoyl, 4-imidazoyl, 2-(1, 3 oxazinyl), 4-(1, 3 oxazinyl), 5-(1, 3 oxazinyl), 2-(thiazinyl), 4-(thiazinyl), 5-(thiazinyl) and 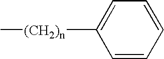
wherein n is 1-3; and
R₃ is hydroxy or —OCONH₂.
